(19) United States
(12) Patent Application Publication
(10) Pub. No.: US 2018/0303429 A1
(43) Pub. Date: Oct. 25, 2018

(54) BLOOD FLOW ANALYZER, BLOOD FLOW ANALYSIS METHOD, AND PROGRAM

(71) Applicant: SEIKO EPSON CORPORATION, Tokyo (JP)

(72) Inventors: Yuta MACHIDA, Kodaira-shi (JP); Kohei YAMADA, Shiojiri-shi (JP)

(73) Assignee: SEIKO EPSON CORPORATION, Tokyo (JP)

(21) Appl. No.: 15/945,882

(22) Filed: Apr. 5, 2018

(30) Foreign Application Priority Data
Apr. 25, 2017 (JP) 2017-086625
Oct. 26, 2017 (JP) 2017-206737

(51) Int. Cl. A61B 5/00; A61B 5/026

(52) U.S. Cl. CPC A61B 5/7217; A61B 5/0261; A61B 5/0075

(57) ABSTRACT

A blood flow analyzer includes a signal processing section which performs filter processing on a detection signal which indicates the intensity of a laser beam having passed through a blood vessel so that a component having a frequency in a predetermined processing band is suppressed in comparison with a component having a frequency which is lower than a frequency at the lower end of the processing band, and an arithmetic processing section which generates information regarding blood flow in the blood vessel from the signal after the filter processing.

### BACKGROUND

### 1. Technical Field

The present invention relates to a technique for generating information regarding a fluid such as blood.

### 2. Related Art

A technique for measuring a blood flow rate in a living body has been proposed. For example, JP-A-2012-210321 (Patent Document 1) discloses a configuration in which a light having passed through a blood vessel of a living body is received by a light receiving element, and the product of the power spectrum of a detection signal which indicates the intensity of the received light by each numerical value of a frequency is integrated in a range of 200 Hz or more and 15 kHz or less, thereby measuring a blood flow rate in a living body.

However, shot noise which is distributed uniformly over a wide range on a frequency axis can be inevitably generated in the detection signal. In the technique disclosed in the Patent Document 1, the product of the power spectrum of a detection signal by each numerical value of a frequency is integrated, and therefore, the shot noise is emphasized more in a higher frequency range. Therefore, the technique has a problem that when the integration range is not strictly selected, the blood flow rate cannot be measured with high accuracy. Although the above description focuses on the measurement of the blood flow rate, the same problem is assumed to occur in a variety of situations where various types of fluids represented by blood are analyzed.

### SUMMARY

An advantage of some aspects of the invention is to analyze a fluid such as blood with high accuracy by reducing the effect of shot noise on the detection signal.

A blood flow analyzer according to a preferred embodiment of the invention includes a signal processing section which performs filter processing on a detection signal which indicates the intensity of a laser beam having passed through a blood vessel so that a component having a frequency in a predetermined processing band is suppressed in comparison with a component having a frequency which is lower than a frequency at the lower end of the processing band, and an arithmetic processing section which generates information regarding blood flow in the blood vessel from the signal after the filter processing. In the above embodiment, filter processing on a detection signal is performed so that a component having a frequency in a predetermined processing band is suppressed in comparison with a component having a frequency which is lower than a frequency at the lower end of the processing band, and therefore, for example, the effect of shot noise which becomes particularly dominant on the high frequency side is reduced. Accordingly, it is possible to generate information regarding blood flow with high accuracy.

In a preferred embodiment of the invention, the arithmetic processing section generates the information regarding blood flow by integrating the product of the intensity at each frequency in the intensity spectrum of the signal after the filter processing by the frequency in a predetermined arithmetic range, and the processing band and the arithmetic range partially overlap each other. In a configuration in which the product of the intensity at each frequency in the intensity spectrum by the frequency (weighted intensity) is integrated, shot noise on the high frequency side in the intensity spectrum is emphasized. According to the preferred embodiment of the invention in which the processing band and the arithmetic range partially overlap each other, even in a case where the arithmetic range is ensured sufficiently wide so as to include a part of the band on the high frequency side where shot noise is dominant in the detection signal, the effect of shot noise on the high frequency side is reduced. Accordingly, it is possible to generate information regarding blood flow with high accuracy.

In a preferred embodiment of the invention, the signal processing section performs the filter processing on the detection signal so that a component having a higher frequency in the predetermined processing band is suppressed more. In the above embodiment, the filter processing on the detection signal is performed so that a component having a higher frequency in the predetermined processing band is suppressed more, and therefore, an advantage of reducing the effect of shot noise which is particularly dominant on the high frequency side can be more effectively realized.

In a preferred embodiment of the invention, the arithmetic range is a range between a first frequency and a second frequency which is higher than the first frequency, and the frequency at the lower end of the processing band is lower than the second frequency. In the above embodiment, the frequency at the lower end of the processing band is lower than the second frequency which is at the upper end of the arithmetic range, and therefore, even in a case where the second frequency is set rather high so that the arithmetic range is sufficiently ensured, the effect of shot noise on the high frequency side is reduced. Accordingly, it is possible to generate information regarding blood flow with high accuracy.

In a preferred embodiment of the invention, the frequency at the lower end of the processing band is higher than a frequency which is higher than the first frequency by ½ of the arithmetic range. In the above embodiment, the frequency at the lower end of the processing band is higher than a frequency which is higher than the first frequency by ½ of the arithmetic range, and therefore, the above-mentioned advantage that a fluid such as blood is analyzed with high accuracy while reducing the effect of shot noise in the detection signal can be more effectively realized.

In a preferred embodiment of the invention, the frequency at the lower end of the processing band is lower than a frequency which is higher than the first frequency by ¾ of the arithmetic range. In the above embodiment, the frequency at the lower end of the processing band is lower than a frequency which is higher than the first frequency by ¾ of the arithmetic range, and therefore, the above-mentioned advantage that a fluid such as blood is analyzed with high accuracy while reducing the effect of shot noise in the detection signal can be more effectively realized.

In a preferred embodiment of the invention, the frequency at the lower end of the processing band is a frequency which is higher than the first frequency by ⅔ of the arithmetic range. In the above embodiment, the frequency at the lower end of the processing band is a frequency which is higher than the first frequency by ⅔ of the arithmetic range, and therefore, the above-mentioned advantage that a fluid such as blood is analyzed with high accuracy while reducing the effect of shot noise in the detection signal can be more effectively realized.

In a preferred embodiment of the invention, the processing band is a range where the degree of suppression of the detection signal by the signal processing section is 6 dB/Oct or more. In the above embodiment, the detection signal is suppressed by a frequency response of 6 dB/Oct in the processing band. Therefore, it is possible to effectively reduce the effect of shot noise which is emphasized due to multiplication of the intensity at each frequency of the intensity spectrum by the frequency.

A blood flow analysis method according to a preferred embodiment of the invention includes performing filter processing on a detection signal which indicates the intensity of a laser beam having passed through a blood vessel so that a component having a frequency in a predetermined processing band is suppressed in comparison with a component having a frequency which is lower than a frequency at the lower end of the processing band, and generating information regarding blood flow in the blood vessel from the signal after the filter processing. In the above embodiment, filter processing on a detection signal is performed so that a component having a frequency in a predetermined processing band is suppressed in comparison with a component having a frequency which is lower than a frequency at the lower end of the processing band, and therefore, for example, the effect of shot noise which becomes particularly dominant on the high frequency side is reduced. Accordingly, it is possible to generate information regarding blood flow with high accuracy.

A program according to a preferred embodiment of the invention causes a computer to function as a signal processing section which performs filter processing on a detection signal which indicates the intensity of a laser beam having passed through a blood vessel so that a component having a frequency in a predetermined processing band is suppressed in comparison with a component having a frequency which is lower than a frequency at the lower end of the processing band, and an arithmetic processing section which generates information regarding blood flow in the blood vessel from the signal after the filter processing. In the above embodiment, filter processing on a detection signal is performed so that a component having a frequency in a predetermined processing band is suppressed in comparison with a component having a frequency which is lower than a frequency at the lower end of the processing band, and therefore, for example, the effect of shot noise which becomes particularly dominant on the high frequency side is reduced. Accordingly, it is possible to generate information regarding blood flow with high accuracy.

### BRIEF DESCRIPTION OF THE DRAWINGS

The invention will be described with reference to the accompanying drawings, wherein like numbers reference like elements.

FIG. 1 is a side view of a blood flow analyzer according to a first embodiment of the invention.

FIG. 2 is a configuration diagram focusing on the function of the blood flow analyzer.

FIG. 3 is a configuration diagram focusing on a light receiving section and an output circuit.

FIG. 4 is a flowchart illustrating an operation of an arithmetic processing section.

FIG. 5 is a frequency response of filter processing by a signal processing section.

FIG. 6 is an explanatory diagram focusing on a frequency at the lower end of the processing band.

FIG. 7 is the intensity spectrum of a detection signal.

FIG. 8 is an explanatory diagram of a problem of shot noise in a comparison example.

FIG. 9 is an explanatory diagram of the advantage of the first embodiment.

FIG. 10 is a diagram for illustrating a method for specifying a frequency at the lower end of the processing band.

FIG. 11 is a diagram showing an example of determining a frequency at the lower end of the processing band.

FIG. 12 is a configuration diagram focusing on a light receiving section and an output circuit in a second embodiment.

FIG. 13 is a schematic diagram showing an example of use of a blood flow analyzer according to a third embodiment.

FIG. 14 is a schematic diagram showing another example of use of the blood flow analyzer according to the third embodiment.

FIG. 15 is a graph showing a frequency spectrum in supplementation on the frequency at the lower end of the processing band.

FIG. 16 is a graph showing a frequency spectrum in supplementation on the frequency at the lower end of the processing band.

FIG. 17 is a graph showing a frequency spectrum in supplementation on the frequency at the lower end of the processing band.

FIG. 18 is a configuration diagram focusing on a light receiving section and an output circuit in a modification example.

FIG. 19 is a frequency response of filter processing by a signal processing section in a modification example.

FIG. 20 is a frequency response of filter processing by a signal processing section in a modification example.

FIG. 21 is a configuration diagram of a blood flow analyzer in a modification example.

FIG. 22 is a configuration diagram of a blood flow analyzer in a modification example.

FIG. 23 is a configuration diagram of a blood flow analyzer in a modification example.

### DESCRIPTION OF EXEMPLARY EMBODIMENTS

### First Embodiment

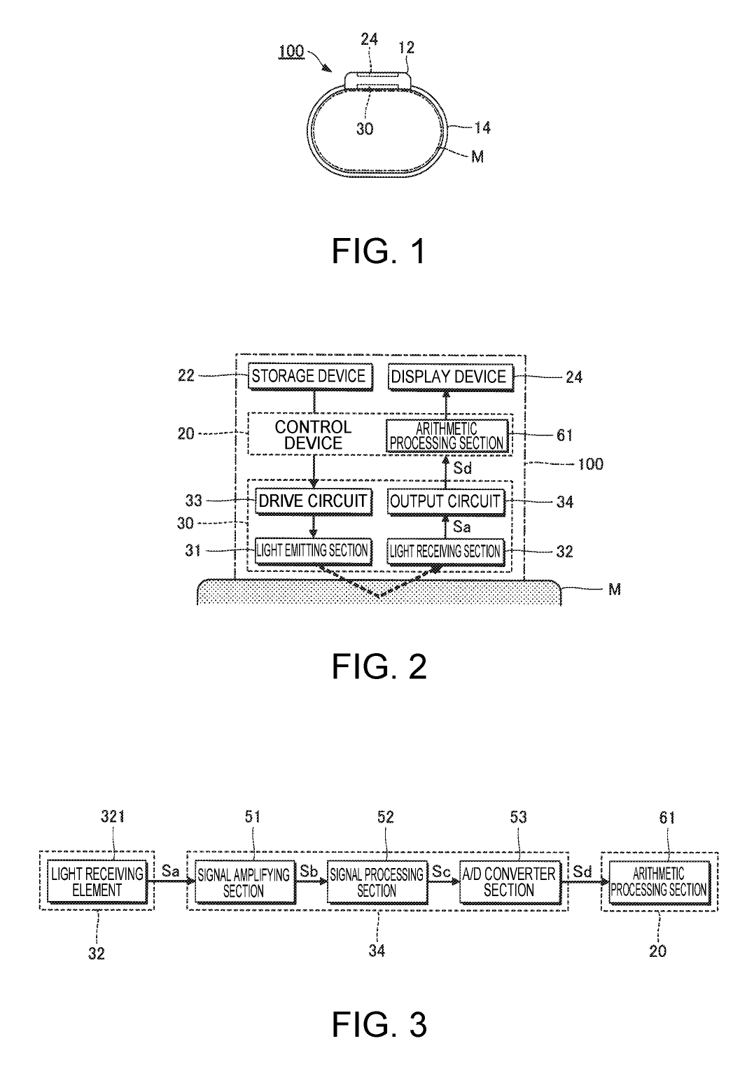

FIG. 1 is a side view of a blood flow analyzer 100 according to a first embodiment of the invention. The blood flow analyzer 100 of the first embodiment is a living body measurement apparatus for noninvasively generating information regarding blood flow in a blood vessel (hereinafter referred to as “blood flow information”) of a test subject (an exemplification of the living body), and is worn on a region which becomes a measurement target (hereinafter referred to as “measurement region”) M of the body of a test subject. As illustrated in FIG. 1, the blood flow analyzer 100 of the first embodiment is a watch-type portable apparatus including a housing section 12 and a belt 14. That is, by winding the belt around the wrist which is an exemplification of the measurement region M, the blood flow analyzer 100 is worn on the wrist of a test subject. As for the blood flow information of the first embodiment, the blood flow velocity (for example, a distance at which red blood cells move through an artery in a unit time) in a test subject is generated as the blood flow information.

FIG. 2 is a configuration diagram focusing on the function of the blood flow analyzer 100. As illustrated in FIG. 2, the blood flow analyzer 100 of the first embodiment includes a control device 20, a storage device 22, a display device 24, and a detection device 30. The control device 20 and the storage device 22 are placed inside the housing section 12. As illustrated in FIG. 1, the display device 24 (for example, a liquid crystal display panel) is placed, for example, on a surface on the opposite side to the measurement region M in the housing section 12, and displays various types of images including a measurement result under the control of the control device 20.

The detection device 30 shown in FIG. 2 is an optical sensor module which generates a detection signal Sd corresponding to the state of the measurement region M. As illustrated in FIG. 2, the detection device 30 of the first embodiment includes a light emitting section 31, a light receiving section 32, a drive circuit 33, and an output circuit 34. The light emitting section 31 and the light receiving section 32 are placed, for example, at a position (typically, a surface in contact with the measurement region M) facing the measurement region M in the housing section 12. It is also possible to place one or both of the drive circuit 33 and the output circuit 34 as an external circuit which is a separate body from the detection device 30.

The light emitting section 31 is a light source which irradiates the measurement region M with a light. The light emitting section 31 of the first embodiment irradiates the measurement region M with a narrow-band coherent laser beam. For example, a light emitting element such as a VCSEL (Vertical Cavity Surface Emitting LASER) which emits a laser beam by resonance in a resonator is preferably used as the light emitting section 31. The light emitting section 31 of the first embodiment irradiates the measurement region M with, for example, a light with a predetermined wavelength λ (λ=800 nm to 1300 nm) in a near-infrared region. The drive circuit 33 shown in FIG. 2 allows the light emitting section 31 to emit a light under the control of the control device 20. It is also possible to use a plurality of light emitting elements which emit lights with different wavelengths as the light emitting section 31. Further, the wavelength λ of the light emitted by the light emitting section 31 is not limited within a near-infrared region.

The light emitted from the light emitting section 31 and incident on the measurement region M is repeatedly reflected and scattered inside the measurement region M, and thereafter is emitted to the housing section 12 side and reaches the light receiving section 32. Specifically, the light having passed through a blood vessel such as an artery (for example, a radial artery or an ulnar artery) present inside the measurement region M and the blood in the blood vessel reaches the light receiving section 32. The light receiving section 32 receives the light coming from the measurement region M. The light receiving section 32 of the first embodiment generates a detection signal Sa which indicates the intensity of the light reaching from the measurement region M. For example, as illustrated in FIG. 3, a light receiving element 321 such as a photo diode (PD) which generates an electrical charge corresponding to the intensity of the received light is preferably used as the light receiving section 32. The detection signal Sa is an analog current signal corresponding to the intensity of the light received from the measurement region M. As understood from the above description, the detection device 30 of the first embodiment is a reflection-type optical sensor in which the light emitting section 31 and the light receiving section 32 are located on one side with respect to the measurement region M.

The light reaching the light receiving section 32 includes a component reflected by a tissue (resting tissue) which rests inside the measurement region M and a component reflected by an object (typically a red blood cell) which moves in an artery inside the measurement region M. The frequency of the light does not shift before and after reflection by a resting tissue, however, the frequency of the light shifts by a shift amount (hereinafter referred to as “frequency shift amount”) Δf in proportion to the moving velocity of the red blood cell (that is, the blood flow velocity) before and after reflection by the red blood cell. That is, the light having passed through the measurement region M and reaching the light receiving section 32 includes a component whose frequency has shifted by the frequency shift amount Δf (frequency shift) from the frequency of the light emitted from the light emitting section 31. The detection signal Sa of the first embodiment is an optical beat signal which reflects the frequency shift by the blood flow inside the measurement region M.

The output circuit 34 shown in FIG. 2 generates a detection signal Sd from the detection signal Sa generated by the light receiving section 32. The detection signal Sd is a digital voltage signal corresponding to the intensity of the light received by the light receiving section 32. As described above, the light irradiated onto the measurement region M passes through the blood vessel and the blood inside the measurement region M and thereafter reaches the light receiving section 32. Therefore, the detection signal Sd can also be referred to as a signal which indicates the intensity of a light having passed through the blood of a test subject.

FIG. 3 is a configuration diagram of the light receiving section 32 and the output circuit 34 in the first embodiment. As illustrated in FIG. 3, the output circuit 34 of the first embodiment is configured to include a signal amplifying section 51, a signal processing section 52, and an A/D converter section 53. The signal amplifying section 51 converts the detection signal Sa supplied from the light receiving section 32 into a voltage signal, and also amplifies the signal, thereby generating a detection signal Sb. For example, the signal amplifying section 51 is configured to include a current/voltage converter circuit which converts the detection signal Sa into a voltage signal and a voltage amplification circuit which amplifies the voltage signal.

The signal processing section 52 generates a detection signal Sc by performing predetermined filter processing on the detection signal Sb (that is, the signal which indicates the intensity of the laser beam having passed through the blood vessel) supplied from the signal amplifying section 51. A specific example of the filter processing performed by the signal processing section 52 will be described later. The A/D converter section 53 converts the analog detection signal Sc generated by the signal processing section 52 into a digital detection signal Sd by a predetermined sampling frequency Fs. As understood from the above description, the detection signals S (Sa, Sb, Sc, and Sd) are optical beat signals which reflect the frequency shift by the blood flow inside the measurement region M.

The control device 20 shown in FIG. 2 is an arithmetic processing device such as a CPU (Central Processing Unit) or an FPGA (Field-Programmable Gate Array) and controls the entire blood flow analyzer 100. The storage device 22 is constituted by, for example, a non-volatile semiconductor memory, and stores a program to be executed by the control device 20 and various types of data to be used by the control device 20. A configuration in which the function of the control device 20 is distributed to a plurality of integrated circuits, or a configuration in which a part or all of the functions of the control device 20 are realized by a dedicated electronic circuit can also be adopted. In FIG. 2, the control device 20 and the storage device 22 are shown as separate elements, however, it is also possible to realize the control device 20 including the storage device 22 by, for example, an ASIC (Application Specific Integrated Circuit) or the like.

The control device 20 of the first embodiment functions as an arithmetic processing section 61 by executing a program (application program) stored in the storage device 22. The arithmetic processing section 61 generates the blood flow information of a test subject from the detection signal Sd generated by the output circuit 34 of the detection device (that is, the signal after processing by the signal processing section 52). The arithmetic processing section 61 of the first embodiment calculates the blood flow velocity in the artery inside the measurement region M as the blood flow information as described above.

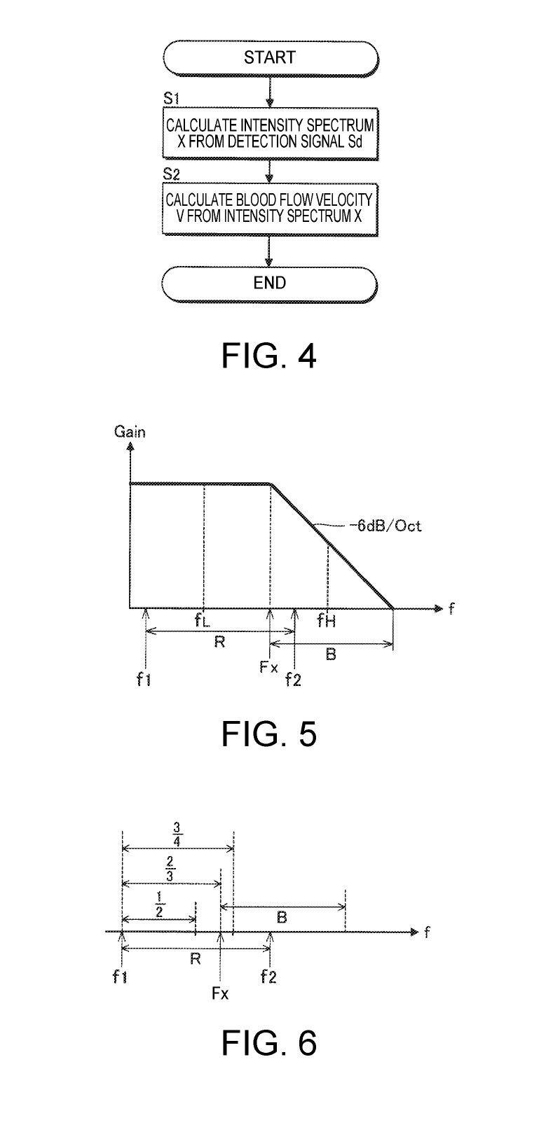

FIG. 4 is a flowchart of processing for calculating the blood flow velocity by the arithmetic processing section 61. For example, the processing shown in FIG. 4 is performed every predetermined period of time. When the processing shown in FIG. 4 is started, the arithmetic processing section 61 calculates an intensity spectrum X from the detection signal Sd (S1). The intensity spectrum X is a distribution of the intensity (power or amplitude) P(f) of a signal component corresponding to each frequency f on a frequency axis in the detection signal Sd. In the calculation of the intensity spectrum X, a known frequency analysis such as discrete Fourier transform can be arbitrarily adopted.

The arithmetic processing section 61 calculates the blood flow velocity V (blood flow information) from the intensity spectrum X of the detection signal Sd (S2). Specifically, the arithmetic processing section 61 of the first embodiment calculates the blood flow rate index F (so-called FLOW value) by arithmetic calculation according to the following numerical formula (1a), and divides the blood flow rate index F by a separately estimated cross-sectional area A of the blood vessel in the measurement region M, thereby calculating the blood flow velocity V (V=F/A). The blood flow rate index F is an index of the blood flow rate (that is, the volume of the blood moving in the artery in a unit time) in the measurement region M. The average of multiple blood flow velocities V calculated at different time points can also be generated by the arithmetic processing section 61 as the blood flow information.

$$F = \frac{{\int }_{f 1}^{f 2} f \times P ( f ) df}{ {I}^{2} } \qquad ( 1 a )$$

The numerical formula (1a) is an arithmetic formula for calculating the blood flow rate index F from each frequency f of the detection signal Sd and the intensity P(f) at the frequency f. The symbol <I²> is an average intensity over the entire band of the detection signal Sd or the intensity P(0) (that is, the signal intensity of a direct current component) at 0 kHz in the intensity spectrum X.

As understood from the numerical formula (1a), the arithmetic processing section 61 of the first embodiment calculates the blood flow rate index F by integrating the product (f×P(f)) of the intensity P(f) at each frequency f in the intensity spectrum X by the frequency f in a predetermined range (hereinafter referred to as “arithmetic range”). The product (f×P(f)) of the intensity P(f) in the intensity spectrum X by the frequency f means the intensity weighted by the frequency f (hereinafter referred to as “weighted intensity”). The arithmetic range corresponds to the integration range of the weighted intensity, and is a range between a predetermined frequency (hereinafter referred to as “lower end frequency”) f1 on a frequency axis and a predetermined frequency (hereinafter referred to as “upper end frequency”) f2 which is higher than the lower end frequency f1. The lower end frequency f1 is an exemplification of the first frequency, and the upper end frequency f2 is an exemplification of the second frequency. As understood from the above description, the arithmetic processing section 61 of the first embodiment generates the blood flow information (blood flow velocity V) from the intensity spectrum X of the detection signal Sd generated by the detection device 30. The display device 24 shown in FIG. 2 displays the blood flow information (blood flow velocity V) generated by the arithmetic processing section 61.

The signal processing section 52 may calculate the blood flow rate index F by arithmetic calculation according to the following numerical formula (1b) in which the integration in the numerical formula (1a) is replaced by sum (Σ).

$$F = \frac{{\sum }_{f = {f}_{1}}^{{f}_{2}} f \cdot Δ f \cdot P ( f )}{ {I}^{2} } \qquad ( 1 b )$$

FIG. 5 is an explanatory diagram of filter processing performed by the signal processing section 52 of the first embodiment. Specifically, the frequency response (that is, the gain distribution in a frequency domain) of filter processing performed by the signal processing section 52 is shown in FIG. 5. As understood from FIG. 5, the signal processing section 52 performs filter processing so that a component having a frequency fH in a predetermined frequency band (hereinafter referred to as “processing band”) B is suppressed in comparison with a component having a frequency fL which is lower than a frequency Fx at the lower end of the processing band B. Specifically, filter processing is performed so that a component having a higher frequency in the processing band B is suppressed more. The signal processing section 52 of the first embodiment performs filter processing in which the gain is continuously decreased so that a component having a higher frequency in the processing band B is suppressed more. For example, a low-pass filter or a band-pass filter is preferably used as the signal processing section 52. Specifically, the signal processing section 52 is constituted by combining a predetermined number of (a single or a plurality of) primary analog filter circuits.

The processing band B is a range where the degree of suppression of the detection signal Sb (that is, the frequency response of filter processing) by the signal processing section 52 is 6 dB/Oct (octave) or more, and is a range on the higher frequency side than the predetermined frequency Fx. That is, in the processing band B, the component having each frequency f constituting the detection signal S is suppressed to a degree not less than the degree (6 dB/Oct) in proportion to the frequency f. For example, when assuming a case where the frequency response of filter processing in the processing band B is set to 6 dB/Oct, the gain at a specific frequency m×f (m is a natural number) in the processing band B is set to 1/m of the gain at the frequency f in the processing band B. The frequency Fx corresponds to the lower end of a range where the degree of suppression of the detection signal Sb is 6 dB/Oct or more (that is, the processing band B).

As illustrated in FIG. 5, the arithmetic range R to be applied to the calculation of the blood flow rate index F and the processing band B of the filter processing by the signal processing section 52 partially overlap each other. Specifically, a portion on the high frequency side in the arithmetic range R and a portion on the low frequency side of the processing band B overlap each other. That is, the frequency Fx at the lower end of the processing band B is higher than the lower end frequency f1 of the arithmetic range R and is equal to or lower than the upper end frequency f2 of the arithmetic range R. In FIG. 5, a case where the frequency Fx at the lower end of the processing band B is lower than the upper end frequency f2 of the arithmetic range R is exemplified.

FIG. 6 is an explanatory diagram focusing on the frequency Fx at the lower end of the processing band B. Specifically, the frequency Fx is higher than a frequency which is higher than the lower end frequency f1 by ½ of the arithmetic range R and lower than the upper end frequency f2. Preferably, the frequency Fx is higher than a frequency which is higher than the lower end frequency f1 by ½ of the arithmetic range R and lower than a frequency which is higher than the lower end frequency f1 by ¾ of the arithmetic range R. In FIG. 6, a case where a frequency which is higher than the first frequency by ⅔ of the arithmetic range R is defined as the frequency Fx is exemplified.

Here, the upper end frequency f2 needs to be the Nyquist frequency (Fs/2) of the A/D converter section 53 or less. That is, the upper end frequency f2 of the arithmetic range R is set to a numerical value between the frequency Fx at the lower end of the processing band B and the Nyquist frequency (Fs/2) of the A/D converter section 53 (Fx≤f2≤Fs/2). For example, when assuming a configuration in which the frequency Fx in the processing band B is 45 kHz and the A/D converter section 53 operates at a sampling frequency Fs of 100 kHz, the upper end frequency f2 is set to an appropriate numerical value (for example, 50 kHz) in a range of 45 kHz (=Fx) or more and 50 kHz (=Fs/2) or less. On the other hand, the lower end frequency f1 is set to a sufficiently small numerical value (for example, about 200 Hz) in comparison with the upper end frequency f2. As understood from the above description, the arithmetic processing section 61 of the first embodiment calculates the blood flow rate index F by integrating the weighted intensity (f×P(f)) in the arithmetic range R including a portion of the processing band B which is suppressed through filter processing performed by the signal processing section 52.

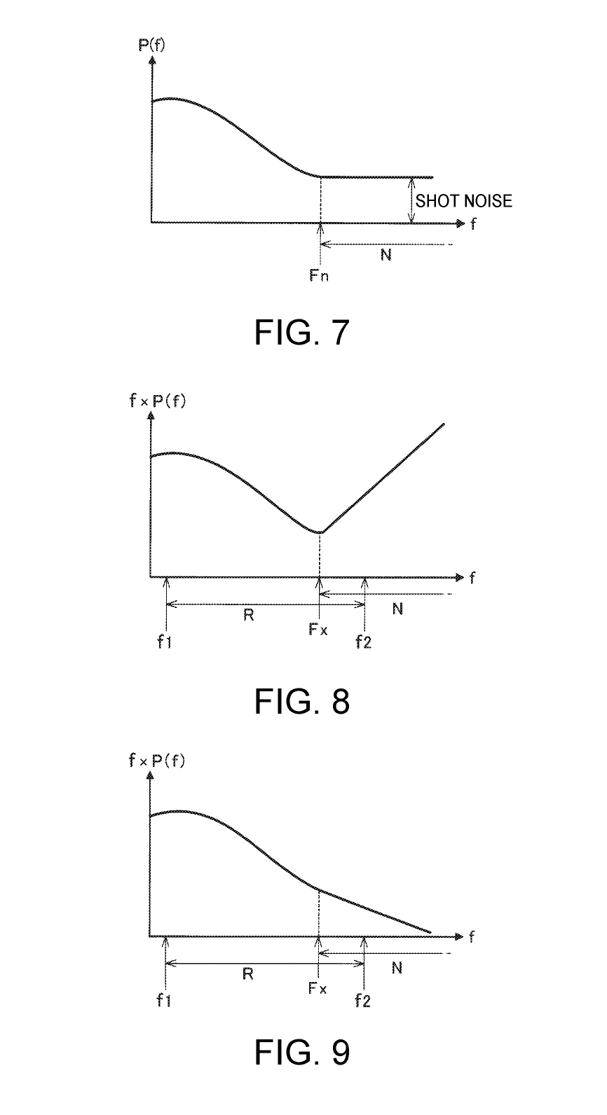

In FIG. 7, the intensity spectrum X of the detection signal Sd is shown. Shot noise inevitably generated due to a circuit element such as the light receiving section 32 or the output circuit 34 is included in the detection signal Sd. Shot noise is white noise uniformly distributed over a wide range of the frequency f. On the other hand, the intensity of a signal component derived from a light having passed through the measurement region M (that is, an original analysis target) tends to be lower on the higher frequency side. Therefore, as understood from FIG. 7, the effect of shot noise on the light having passed through the measurement region M is dominant in the frequency band N on the high frequency side in the intensity spectrum X. A frequency Fn at the lower end of the frequency band N in which the effect of shot noise is dominant (hereinafter referred to as “high noise frequency”) can shift according to the state of the blood vessel or the blood in the measurement region M.

FIGS. 8 and 9 each show the distribution of the weighted intensity (f×P(f)) on the frequency axis. FIG. 8 shows the distribution of the weighted intensity in a configuration in which filter processing by the signal processing section 52 is omitted (hereinafter referred to as “comparison example”). FIG. 9 shows the distribution of the weighted intensity in the first embodiment in which the signal processing section 52 performs filter processing so that a signal component in the processing band B is suppressed.

From the viewpoint that the blood flow rate index F is measured with high accuracy by arithmetic calculation according to the numerical formula (1a), it is necessary to ensure the arithmetic range R wide. That is, the upper end frequency f2 is required to be set rather high. On the other hand, by multiplication of the intensity P(f) in the intensity spectrum X by the frequency f, in the comparison example, as understood from FIG. 8, shot noise on the high frequency side in the intensity spectrum X is emphasized. Therefore, in a configuration in which the upper end frequency f2 is set rather high, shot noise is dominant on the high frequency side in the arithmetic range R, and as a result, the highly accurate measurement of the blood flow rate index F is inhibited. In a case where the upper end frequency f2 is set rather low in order to solve the above problem, the accuracy of the measurement of the blood flow rate index F may be deteriorated instead as a result of excessive narrowing of the arithmetic range R. As understood from the above description, in order to always measure the blood flow rate index F with high accuracy, it is necessary to shift the upper end frequency f2 according to the state of the blood vessel or the blood in the measurement region M. For example, in a case where the high-noise frequency Fn is high, the upper end frequency f2 is set rather high, and in a case where the high-noise frequency Fn is low, the upper end frequency f2 is set rather low, and so on.

In contrast to the comparison example described above, in the first embodiment, a signal component in the processing band B which partially overlaps the arithmetic range R in the detection signal Sb is suppressed by filter processing. That is, as understood from FIG. 9, regardless of the magnitude of the upper end frequency f2, the effect of shot noise to be emphasized by multiplication of the intensity P(f) by the frequency f is reduced by filter processing in the arithmetic range R. Therefore, for example, even in a configuration in which the upper end frequency f2 is set rather high so as to sufficiently ensure the arithmetic range R, it is possible to measure the blood flow rate index F with high accuracy by reducing the effect of shot noise of the detection signal Sb. Further, since the above-mentioned processing for shifting the upper end frequency f2 according to the state of the blood vessel or the blood in the measurement region M is not needed, there is also an advantage that a load for generating the blood flow information is reduced.

Next, a condition which is established in the above-mentioned state where the arithmetic range R and the processing band B partially overlap each other will be described. A case where the range of the blood flow velocity V in the specification measurable by the blood flow analyzer 100 is a range from the minimum value V1 to the maximum value V2 is assumed. As described above, the frequency shift amount Δf attributed to light reflection by a red blood cell in the blood is in proportion to the blood flow velocity V. Specifically, the frequency shift amount Δf is represented by the following numerical formula (2).

$$Δ f = \frac{2 n \cdot cos θ}{λ} V \qquad ( 2 )$$

The symbol λ in the numerical formula (2) is the wavelength of the light irradiated onto the measurement region M by the light emitting section 31, and the symbol θ is an incident angle of the light incident on the measurement region M from the light emitting section 31. When assuming the actual blood flow analyzer 100, the wavelength λ is known as the wavelength of the light emitted by the light emitting section 31, and the incident angle θ is determined from the angle of the optical axis of the light emitting section 31 with respect to the surface of the measurement region M. The symbol n is the refractive index of the measurement region M (particularly, artery and blood) and is a known numerical value in a range of approximately 1.33 to 1.34. When the maximum value V2 of the blood flow velocity V which is assumed to be measured by the blood flow analyzer 100 is substituted into the numerical formula (2) along with the above-mentioned respective constants (λ, θ, and n), the maximum frequency shift amount Δf2 in the range measurable by the blood flow analyzer 100 can be obtained.

The maximum value V2 of the blood flow velocity V can be measured using a flow velocity meter using ultrasound. In a case where an arterial blood flow is measured, for example, the maximum value V2 of the blood flow velocity V is known to be 0.8 m/sec or more and 1.2 m/sec or less, and in a case where a capillary blood flow is measured, for example, the maximum value V2 of the blood flow velocity V is known to be 2 mm/sec or more and 12 mm/sec or less.

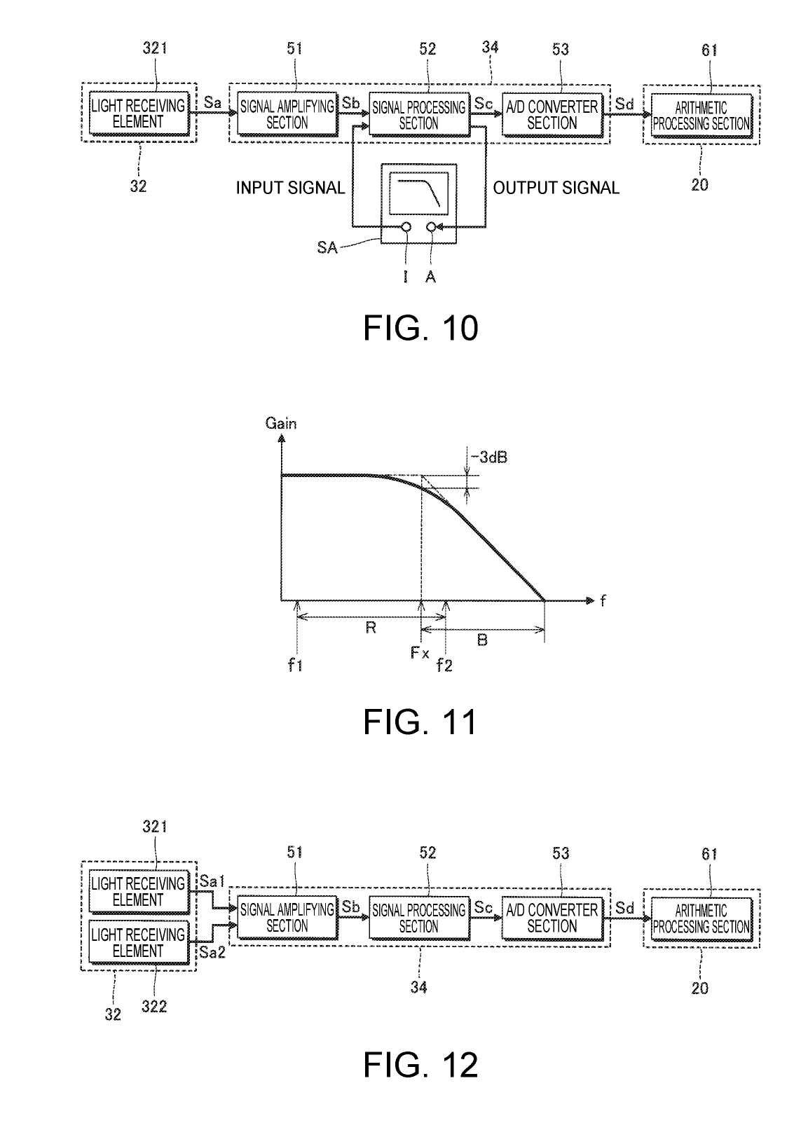

Next, a method for determining the lower end frequency Fx in the processing band B will be described. FIG. 10 is a diagram for illustrating a method for specifying the lower end frequency Fx in the processing band B. As illustrated in FIG. 10, for example, a sinewave input signal is input to the signal processing section 52 from a terminal I of a spectrum analyzer SA, and an electrical power or an electrical voltage from the signal processing section 52 is input to a terminal A as an output signal. Here, by changing the frequency of the input signal and acquiring the output signal for each frequency, the frequency response as illustrated in FIGS. 5 to 9 can be obtained. The lower end frequency Fx in the processing band B is a frequency at which the output signal decreases as the frequency of the input signal increases. Specifically, as shown in FIG. 11, a frequency at which the output signal decreases by 3 dB as the frequency of the input signal increases is defined as the lower end frequency Fx in the processing band B. In FIG. 10, a case where the frequency response is obtained from the relationship between the input signal and the output signal of the signal processing section 52 is exemplified, however, the input signal may be input to the signal amplifying section 51, or the output signal may be acquired from an element (for example, the A/D converter section 53) downstream of the signal processing section 52.

In order to obtain more blood flow information, it is desired to obtain blood flow information up to the maximum value V2 of the blood flow velocity V. In other words, it is desired that the upper end frequency f2 be equal to or higher than the frequency shift amount Δf2 (f2≥Δf2). In this case, when the relationship that the frequency shift amount Δf2 is higher than the frequency Fx (Fx<Δf2) can be confirmed, the upper end frequency f2 is equal to or higher than the frequency shift amount Δf2 (Δf2≤f2), and therefore, the upper end frequency f2 is higher than the frequency Fx (Fx<f2). The fact that the upper end frequency f2 is higher than the frequency Fx (Fx<f2) means that a condition in which the arithmetic range R and the processing band B partially overlap each other is established.

### Second Embodiment

A second embodiment of the invention will be described. In each embodiment to be exemplified below, elements having the same operation or function as in the first embodiment are denoted by the same reference numerals used in the description of the first embodiment, and the detailed description thereof will be omitted as appropriate.

FIG. 12 is a configuration diagram of a light receiving section 32 and an output circuit 34 in the second embodiment. As illustrated in FIG. 12, the light receiving section 32 of the second embodiment is configured to include a light receiving element 321 and a light receiving element 322 placed at different positions. The light receiving element 321 generates a detection signal Sa1 corresponding to the intensity of the light received from the measurement region M, and the light receiving element 322 generates a detection signal Sa2 corresponding to the intensity of the light received from the measurement region M.

A signal amplifying section 51 of the second embodiment generates a detection signal Sb which corresponds to a difference between the detection signal Sa1 generated by the light receiving element 321 and the detection signal Sa2 generated by the light receiving element 322. Therefore, the detection signal Sb in which steady noise included commonly in the detection signal Sa1 and the detection signal Sa2 has been reduced is generated. For example, a differential amplifier circuit is preferably used as the signal amplifying section 51. A signal processing section 52 performs the same filter processing as in the first embodiment on the detection signal Sb supplied from the signal amplifying section 51. The functions and operations of the other elements are the same as in the first embodiment.

In also the second embodiment, the same advantage as that of the first embodiment is realized. Further, in the second embodiment, the detection signal Sb which corresponds to a difference between the detection signal Sa1 generated by the light receiving element 321 and the detection signal Sa2 generated by the light receiving element 322 is generated. That is, the detection signal Sb in which noise included commonly in the detection signal Sa1 and the detection signal Sa2 has been reduced and thus the S/N ratio is high is generated. Therefore, the advantage that the blood flow information can be generated with high accuracy is especially remarkable.

### Third Embodiment

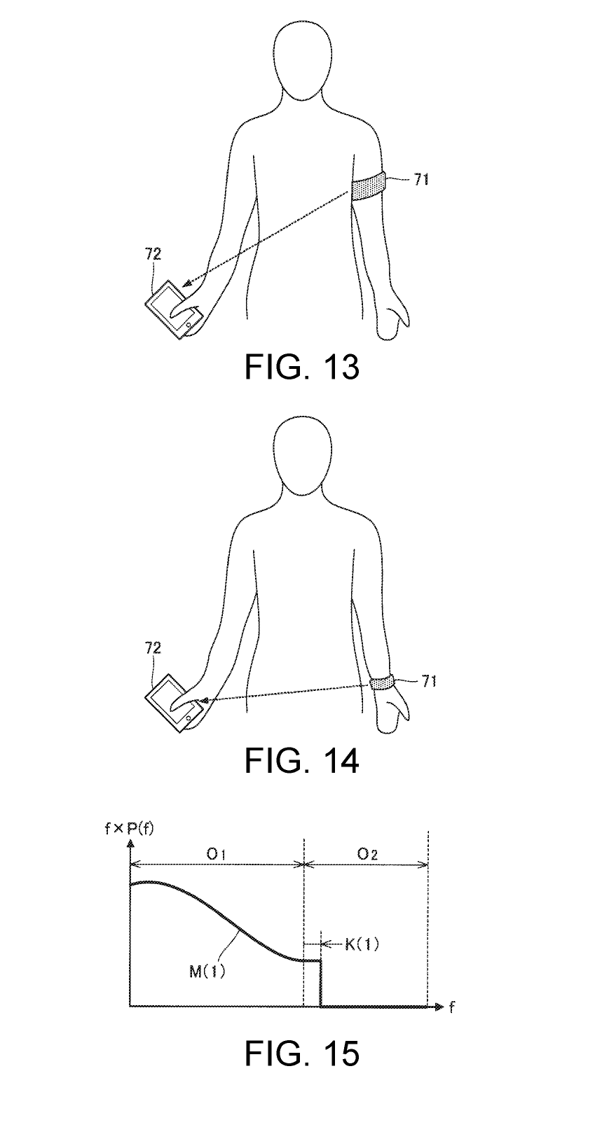

FIG. 13 is a schematic diagram showing an example of use of a blood flow analyzer 100 according to a third embodiment. As illustrated in FIG. 13, the blood flow analyzer 100 includes a detection unit 71 and a display unit 72 which are constituted by mutually separate bodies. The detection unit 71 includes a detection device 30 exemplified in each embodiment described above. In FIG. 13, the detection unit 71 in the form in which it is worn on the upper arm of a test subject is illustrated. As illustrated in FIG. 14, the detection unit 71 in the form in which it is worn on the wrist of a test subject is also preferred.

The display unit 72 includes a display device 24 exemplified in each embodiment described above. For example, an information terminal such as a portable phone or a smartphone is a preferred example of the display unit 72. However, a specific form of the display unit 72 is arbitrary. For example, a test subject may use a watch-type information terminal which can be carried by a test subject or a dedicated information terminal of the blood flow analyzer 100 may be used as the display unit 72.

An arithmetic processing section 61 is, for example, mounted on the display unit 72. A detection signal Sd generated by the detection device 30 of the detection unit 71 is transmitted to the display unit 72 through wired or wireless connection. The arithmetic processing section 61 in the display unit 72 calculates blood flow information from the detection signal Sd and displays the information on the display device 24.

The arithmetic processing section 61 may be mounted on the detection unit 71. The arithmetic processing section 61 calculates blood flow information from the detection signal Sd generated by the detection device 30 and transmits data for displaying the blood flow information to the display unit 72 through wired or wireless connection. The display device 24 of the display unit 72 displays the blood flow information represented by the data received from the detection unit 71.

### Supplementation on Frequency Fx

As exemplified in each embodiment described above, a configuration in which filter processing is performed so that a component having a higher frequency in the processing band B is suppressed more (hereinafter referred to as “configuration A”) is adopted as a preferred embodiment of the invention. A behavior which can be observed from an actual blood flow analyzer (hereinafter referred to as “actual product”) by adopting the configuration A will be described below.

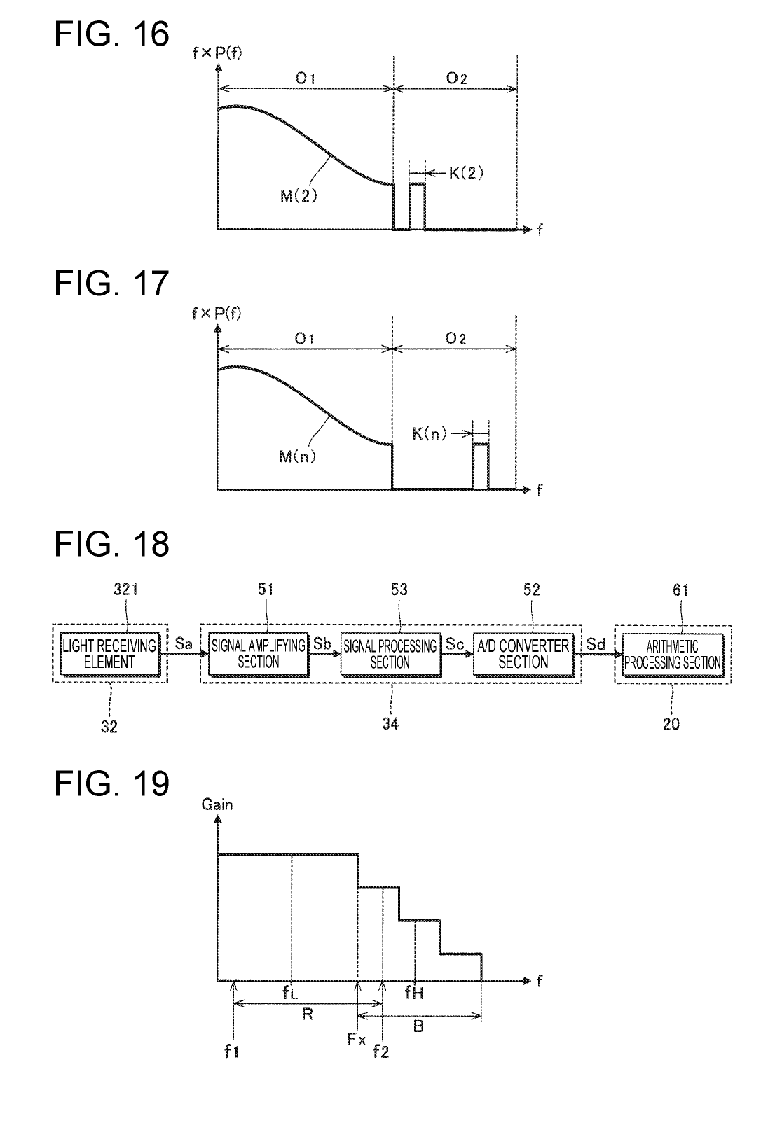

First, N types of frequency spectra M(1) to M(N) are assumed. Each of the frequency spectra M(1) to M(N) corresponds to a product of the frequency f by the intensity spectrum P(f). As shown in FIGS. 15 to 17, a first range O1 and a second range O2 are defined in the frequency domain. The second range O2 is located on the higher frequency side than the first range O1. The second range O2 is divided into N pieces of bands K(1) to K(N). The bands K(1) to K(N) have the same bandwidth. An arbitrary one frequency spectrum M(n) includes a component in the first range O1 and a component in an n-th band K(n) (n=1 to N) in the N pieces of bands K(1) to K(N) in the second range O2. The intensity of the component in the band K(n) is shared among the N pieces of frequency spectra M(1) to M(N). The frequency spectrum M(n) is 0 in a band other than in the band K(n) in the second range O2. The shape of the frequency spectrum M(n) in the first range O1 is arbitrary, but is shared among the N pieces of frequency spectra M(1) to M(N). One arbitrary input signal Y(n) is a signal in a time domain when the intensity distribution in the frequency domain is the frequency spectrum M(n). That is, the input signal Y(n) is generated by inverse Fourier transform of the frequency spectrum M(n). In the generation of the input signal Y(n), for example, a signal generator such as a pulse generator is used.

A case where N types of input signals Y(1) to Y(N) are input sequentially to a wiring or a terminal to which the detection signal Sb is supplied in the actual product is assumed. In a case where an input signal Y(n) is supplied to the actual product, the blood flow rate index F(n) is displayed. As described above, the intensity of the component in the band K(n) is shared among the N types of input signals Y(1) to Y(N). In a case where the actual product adopts the configuration A, a component on the higher frequency side in the processing band B is suppressed more, and therefore, as the band K(n) of the input signal Y(n) is located on the higher frequency side, the blood flow rate index F(n) is smaller numerical value. Therefore, in a case where the following relational formula is satisfied: “blood flow rate index F(1)>blood flow rate index F(2)> . . . >blood flow rate index F(N)” (that is, a case where a component having a higher frequency is suppressed more), it can be determined that the configuration A is adopted.

The above description focuses on the blood flow rate index F, however, the blood flow index for determining whether or not the actual product adopts the configuration A is not limited to the above-mentioned exemplification. For example, various blood flow indices such as an average blood pressure, a pulse pressure, or a blood mass index may be used. Further, in a case where a software filter is used in the actual product, the blood flow rate index F(n) calculated by allowing the light receiving section which generates the detection signal Sa to receive a light so as to generate the input signal Y(n) may be used for determining whether or not the configuration A is adopted in the actual product.

### Modification Example

The respective embodiments exemplified above can be variously modified. Specific modified embodiments will be exemplified below. It is also possible to appropriately combine two or more embodiments arbitrarily selected from the embodiments exemplified below.

(1) It is also possible to realize the blood flow analyzer 100 by a plurality of apparatuses constituted by mutually separate bodies. For example, it is also possible to realize the arithmetic processing section 61 exemplified in each embodiment described above by a general-purpose information terminal such as a portable phone or a smartphone. Further, a configuration in which the blood flow information generated by the arithmetic processing section 61 is displayed on the display device 24 included in the information terminal can also be adopted.

(2) The order of the plurality of elements constituting the output circuit 34 is not limited to the exemplification of each embodiment described above. For example, in each embodiment described above, the detection signal Sc generated by the signal processing section 52 is A/D converted by the A/D converter section 53, however, for example, as illustrated in FIG. 18, it is also possible to reverse the order of the signal processing section 52 and the A/D converter section 53. In the configuration shown in FIG. 18, the A/D converter section 53 converts the detection signal Sb amplified by the signal amplifying section 51 from analog to digital, and the signal processing section 52 performs filter processing on the detection signal Sc after conversion and generates the detection signal Sd. Therefore, a digital filter which suppresses a component in the processing band B in the detection signal Sc is used as the signal processing section 52. It is also possible to realize the signal processing section 52 by allowing the control device 20 to execute a program. That is, the signal processing section 52 may be a software filter.

(3) In each embodiment described above, the blood flow velocity V is exemplified as the blood flow information, however, the type of information regarding blood flow (blood flow information) is not limited to the above-mentioned exemplification. For example, it is also possible to show a blood flow rate index F calculated according to the above numerical formula (1a) as the blood flow information to a test subject. Alternatively, a blood mass index (a so-called MASS value) is calculated from the detection signal Sd, and the blood mass index may be calculated as the blood flow information. It is also possible to generate another living body information from the blood flow information such as the blood flow rate index F, a blood mass index and the blood flow velocity V. For example, various living body information such as a blood pressure, an average blood pressure, a pulse pressure, an oxygen saturation level (SpO2), a blood vessel diameter, and a blood vessel age (blood vessel hardness) can be estimated from the blood flow information such as the blood flow rate index F and the blood flow velocity V.

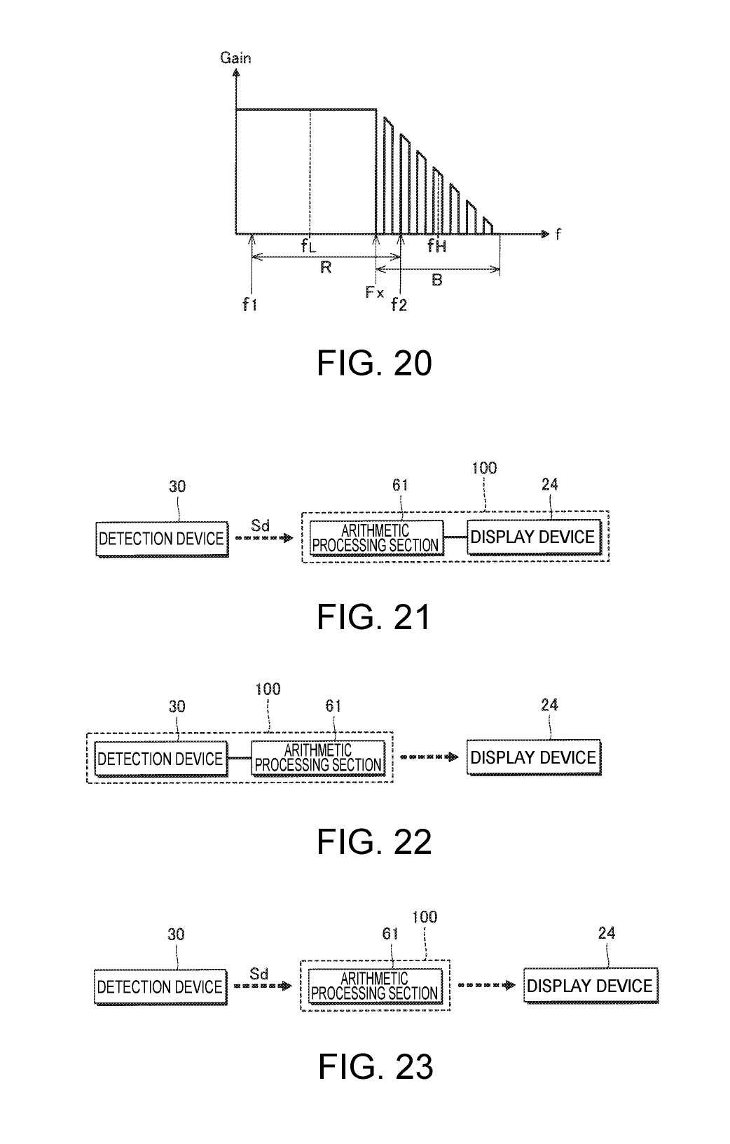

(4) In each embodiment described above, filter processing in which the gain is continuously decreased so that a component having a higher frequency in the processing band B is suppressed more (FIG. 5) is performed, however, the frequency response of the filter processing performed by the signal processing section 52 is not limited to the above-mentioned exemplification. As shown in FIG. 19, filter processing in which the gain is decreased in stages toward the high frequency side in the processing band B may be performed. The filter processing shown in FIGS. 5 and 19 is filter processing such that a component having a higher frequency in the processing band B is suppressed more (that is, filter processing in which as the frequency is higher, the gain monotonically decreases more). Further, as shown in FIG. 20, in the filter processing shown in FIG. 5, ranges in which the gain is set to a specific value may be provided at predetermined intervals in the processing band B. In FIG. 20, a case where ranges in which the gain is set to 0 are provided at predetermined intervals is illustrated, however, the gain to be set may be 0 or more.

Moreover, a plurality of types of filter processing may be combined. For example, a plurality of (two or three) types of filter processing selected from filter processing in which the gain is continuously decreased so that a component having a higher frequency in the processing band B is suppressed more (FIG. 5), filter processing in which the gain is decreased in stages toward the high frequency side (FIG. 19), and filter processing in which ranges in which the gain is set to 0 are provided at predetermined intervals (FIG. 20) may be performed. As understood from the above description, the frequency response of the filter processing is arbitrary as long as the filter processing is performed so that a component having a frequency fH in the processing band B is suppressed in comparison with a component having a frequency fL which is lower than a frequency Fx at the lower end of the processing band B. However, according to each embodiment described above in which filter processing in which the gain continuously shifts such that a component having a higher frequency in the processing band B is suppressed more (FIG. 5) is performed, an advantage that the effect of shot noise which becomes particularly dominant on the high frequency side is reduced can be effectively realized.

(5) In each embodiment described above, the blood flow analyzer 100 constituted as a single apparatus is exemplified, however, as exemplified below, the plurality of elements of the blood flow analyzer 100 can be realized as mutually separate devices.

In each embodiment described above, the blood flow analyzer 100 including the detection device 30 is exemplified, however, as illustrated in FIG. 21, a configuration in which the detection device 30 is provided as a separate body from the blood flow analyzer 100 is also assumed. The detection device 30 is, for example, a portable optical sensor module to be worn on the measurement region M such as the wrist or upper arm of a test subject. The blood flow analyzer 100 is realized by, for example, an information terminal such as a portable phone or a smartphone. The blood flow analyzer 100 may be realized by a watch-type information terminal. The detection signal Sd generated by the detection device 30 is transmitted to the blood flow analyzer 100 through wired or wireless connection. The arithmetic processing section 61 of the blood flow analyzer 100 calculates blood flow information from the detection signal Sd and displays the information on the display device 24. As understood from the above description, the detection device 30 can be omitted from the blood flow analyzer 100.

In each embodiment described above, the blood flow analyzer 100 including the display device 24 is exemplified, however, as illustrated in FIG. 22, a configuration in which the display device 24 is provided as a separate body from the blood flow analyzer 100 is also assumed. The arithmetic processing section 61 of the blood flow analyzer 100 calculates blood flow information from the detection signal Sd and transmits data for displaying the index to the display device 24. The display device 24 may be a dedicated display device, but may be mounted on, for example, an information terminal such as a portable phone or a smartphone, or a watch-type information terminal which can be carried by a test subject. The blood flow information calculated by the arithmetic processing section 61 of the blood flow analyzer 100 is transmitted to the display device 24 through wired or wireless connection. The display device 24 displays the blood flow information received from the blood flow analyzer 100. As understood from the above description, the display device 24 can be omitted from the blood flow analyzer 100.

As illustrated in FIG. 23, a configuration in which the detection device 30 and the display device 24 are provided as separate bodies from the blood flow analyzer 100 (living body analysis section) is also assumed. For example, the blood flow analyzer 100 (living body analysis section) is mounted on an information terminal such as a portable phone or a smartphone.

In the configuration in which the detection device 30 and the blood flow analyzer 100 are provided as separate bodies, it is also possible to mount an element which calculates an intensity spectrum on the detection device 30. The intensity spectrum calculated by the detection device 30 is transmitted to the blood flow analyzer 100 through wired or wireless connection.

(6) In each embodiment described above, the watch-type blood flow analyzer 100 constituted by the housing section 12 and the belt 14 is exemplified, however, a specific form of the blood flow analyzer 100 is arbitrary. For example, the blood flow analyzer 100 in an arbitrary form such as a patch type which can be attached to the body of a test subject, an earring type which can be worn on the auricle of a test subject, a finger-worn type (for example, a nail-worn type) which can be worn on a finger tip of a test subject, a head-mounted type which can be mounted on the head of a test subject, or the like can be adopted.

(7) In each embodiment described above, the blood flow information of a test subject is displayed on the display device 24, however, a configuration for notifying a test subject of the blood flow information is not limited to the above exemplification. For example, it is also possible to notify a test subject of the blood flow information by sound. In the blood flow analyzer 100 of an ear-worn type which can be worn on an ear part of a test subject, a configuration in which the blood flow information is notified by sound is particularly preferred. Notification of a test subject of the blood flow information is not essential. For example, the blood flow information calculated by the blood flow analyzer 100 may be transmitted to another communication device through a communication network. Further, the blood flow information may be stored in the storage device 22 of the blood flow analyzer 100 or a portable recording medium which can be attached to and detached from the blood flow analyzer 100.

(8) In each embodiment described above, the blood flow analyzer 100 which analyzes the blood flow of a test subject is exemplified, however, the range to which the invention is applied is not limited to an analysis of blood flow. For example, it is also possible to apply the invention to a device which analyzes the flow of various types of liquids other than blood (for example, a medicinal liquid which flows in a tube). As understood from the above description, a preferred embodiment of the invention is a device which analyzes a fluid (a fluid analyzer), and the blood flow analyzer 100 explained in each embodiment described above is an exemplification of the fluid analyzer according to a preferred embodiment of the invention.

(9) The blood flow analyzer 100 according to each embodiment described above is realized by cooperation of the control device 20 and the program as exemplified above. The program according to a preferred embodiment of the invention is provided in the form of being stored in a computer readable recording medium and can be installed on a computer. Further, it is also possible to provide the program stored in a recording medium included in a distribution server in a distribution form through a communication network. The recording medium is, for example, a non-transitory recording medium, and is preferably an optical recording medium (optical disk) such as a CD-ROM, but can include a recording medium in an arbitrary known form such as a semiconductor recording medium or a magnetic recording medium. The non-transitory recording medium includes arbitrary recording media excluding transitory propagating signals, and does not exclude volatile recording media.

The entire disclosure of Japanese Patent Applications No. 2017-086625 and No. 2017-206737 are hereby incorporated herein by reference.

What is claimed is:

1. A blood flow analyzer, comprising:
a signal processing section which performs filter processing on a detection signal which indicates the intensity of a laser beam having passed through a blood vessel so that a component having a frequency in a predetermined processing band is suppressed in comparison with a component having a frequency which is lower than a frequency at the lower end of the processing band; and
an arithmetic processing section which generates information regarding blood flow in the blood vessel from the signal after the filter processing.

2. The blood flow analyzer according to claim 1, wherein
the arithmetic processing section generates the information regarding blood flow by integrating the product of the intensity at each frequency in the intensity spectrum of the signal after the filter processing by the frequency in a predetermined arithmetic range, and
the processing band and the arithmetic range partially overlap each other.

3. The blood flow analyzer according to claim 1, wherein the signal processing section performs the filter processing on the detection signal so that a component having a higher frequency in the predetermined processing band is suppressed more.

4. The blood flow analyzer according to claim 2, wherein
the arithmetic range is a range between a first frequency and a second frequency which is higher than the first frequency, and
the frequency at the lower end of the processing band is lower than the second frequency.

5. The blood flow analyzer according to claim 4, wherein the frequency at the lower end of the processing band is higher than a frequency which is higher than the first frequency by ½ of the arithmetic range.

6. The blood flow analyzer according to claim 5, wherein the frequency at the lower end of the processing band is lower than a frequency which is higher than the first frequency by ¾ of the arithmetic range.

7. The blood flow analyzer according to claim 6, wherein the frequency at the lower end of the processing band is a frequency which is higher than the first frequency by ⅔ of the arithmetic range.

8. The blood flow analyzer according to claim 1, wherein the processing band is a range where the degree of suppression of the detection signal by the signal processing section is 6 dB/Oct or more.

9. A blood flow analysis method, comprising:
performing filter processing on a detection signal which indicates the intensity of a laser beam having passed through a blood vessel so that a component having a frequency in a predetermined processing band is suppressed in comparison with a component having a frequency which is lower than a frequency at the lower end of the processing band; and
generating information regarding blood flow in the blood vessel from the signal after the filter processing.

10. A program for causing a computer to function as
a signal processing section which performs filter processing on a detection signal which indicates the intensity of a laser beam having passed through a blood vessel so that a component having a frequency in a predetermined processing band is suppressed in comparison with a component having a frequency which is lower than a frequency at the lower end of the processing band, and
an arithmetic processing section which generates information regarding blood flow in the blood vessel from the signal after the filter processing.
